(19) United States
(12) Patent Application Publication
(10) Pub. No.: US 2021/0378516 A1
(43) Pub. Date: Dec. 9, 2021

(54) METHOD AND SYSTEM FOR POSITIONING INVASIVE MEDICAL TOOLS RELATIVE TO 3D IMAGERY

(71) Applicant: UNIVERSITY OF FLORIDA RESEARCH FOUNDATION, INCORPORATED, Gainesville, FL (US)

(72) Inventors: Brian C. LOBO, Gainesville, FL (US); Marino LEON, Gainesville, FL (US); Reordan O. DEJESUS, Gainesville, FL (US)

(21) Appl. No.: 17/283,678

(22) PCT Filed: Oct. 8, 2019
(86) PCT No.: PCT/US2019/055158

§ 371 Date: Apr. 8, 2021

Related U.S. Application Data
(60) Provisional application No. 62/742,786, filed on Oct. 8, 2018.

(51) Int. Cl. A61B 5/00; A61B 1/015; A61B 5/06

(52) U.S. Cl. CPC A61B 5/0036; A61B 5/0062; A61B 2090/3937; A61B 5/062; A61B 5/702; A61B 1/015

(57) ABSTRACT

A method and apparatus for invasive medical procedures uses a tool that includes an operative portion and a stem having a distal end attached to a proximal end of the operative portion and a proximal end configured to be held outside a subject during the invasive medical action. The tool can include a recess along the stem and a viewport. The recess is configured to removably engage an electromagnetic tracking system component comprising an electromagnetic sensor and an insulated electrical wire connected to the electromagnetic sensor. The viewport is configured to reveal a portion of the recess where the electromagnetic sensor is to be disposed. Alternatively, a frame is rigidly and removably attached to a proximal portion of the tool. The frame is also rigidly attached to electromagnetic tracking sensor suite that provides position and orientation of the frame with six degrees of freedom.

### BACKGROUND

Image-guided therapy (IGT) is based on the registration of pre-operative (e.g., computed tomography, CT) or intra-operative (e.g., ultrasound, US) patient imagery to the actual operative field inside the patient for surgical intervention. Thus, IGT provides freehand navigation or guidance for mechatronic positioning of medical instruments. These methods commonly rely on the localization of surgical equipment with respect to the patient and the imagery. This localization in 3D space is referred to as tracking, and is a key enabling technology for computer assisted interventions.

Electromagnetic (EM) tracking localizes small sensors inside a magnetic field of known geometry, which is created by a field generator (FG). Different small sensors are commercially available, such as sensors for the Ascension dc tracking system, for the Polhemus ac tracking system, a Six DoF Sensor for the NDI Aurora ac tracking system, and a Passive EM transponder of the Calypso GPS for the Body system, referred to as a beacon. Several of these sensors are a few millimeters in length and connected by small gauge insulated wire to an electronic tracking base system.

In general, image guided therapy involves the spatial registration of a 3D medical image for the patient and the EM tracking system with the same spatial fiducials. The EM tracking sensor is then located relative to the 3D medical image, with accuracy of 1 to 5 millimeters.

### SUMMARY

Techniques are provided for adapting invasive medical tools to removably receive an EM sensor for positioning an operational portion of the tool relative to 3D medical imagery. As used herein, the term invasive medical tool refers to any tool used during superficial, subcutaneous and deep medical procedures including surgical biopsies, surgical resections, image guided biopsies and implant placement.

In a first set of embodiments, an invasive medical tool includes: an operative portion that is configured to perform some invasive medical action. The invasive medical tool also includes a stem having a distal end attached to a proximal end of the operative portion and a proximal end configured to be held outside a subject during the invasive medical action. The invasive medical tool further includes a recess along the stem. The recess is configured to removably engage an electromagnetic tracking system component comprising an electromagnetic sensor and an insulated electrical wire connected to the electromagnetic sensor. The invasive medical tool still further includes a viewport configured to reveal a portion of the recess where the electromagnetic sensor is to be disposed.

In a second set of embodiments, a system includes the invasive medical tool of the first set of embodiments and the electromagnetic tracking system comprising a magnetic field generator configured to be disposed outside a subject into which the tool is to be inserted and the electromagnetic sensor and the insulated electrical wire, wherein the sensor and a portion of the insulated electrical wire are removably disposed inside the recess.

In some embodiments of the second set, the system includes a brace configured to hold motionless two portions of the subject on opposite sides of a joint of the subject.

In a third set of embodiments, a method for guided surgical procedures includes producing three dimensional imagery of a subject relative to spatial fiducials and registering an electromagnetic (EM) tracking system relative to the spatial fiducials. The method further includes inserting a wired sensor and wire of the EM tracking system into a first recess with a first viewport of a first invasive medical tool until the wired sensor is disposed at a desired location and evident through the viewport. The method also includes using the first invasive medical tool in the subject while tracking the position of the wired sensor relative to the three-dimensional imagery of the subject based on the EM tracking system and the spatial fiducials.

In some embodiments of the third set, the method also includes selecting a different second invasive medical tool and removing the wired sensor and wire of the EM tracking system from the first recess of the first invasive medical tool. The method yet further includes inserting the wired sensor and wire of the EM tracking system into a second recess with a second viewport of the second invasive medical tool until the wired sensor is disposed at a desired location and evident through the second viewport. This embodiment even further includes using the second invasive medical tool in the subject while tracking the position of the wired sensor relative to the three-dimensional imagery of the subject based on the EM tracking system and the spatial fiducials.

In some embodiments of the third set, the method still further includes, before producing three-dimensional imagery of the subject, attaching a brace to the subject to immobilize a joint of the subject. The method also includes, before producing the imagery, attaching at least one spatial fiducial on each of at least two different sides of the joint.

In a fourth set of embodiments, an apparatus for guided surgical procedures includes a rigid invasive medical tool. A distal end of the tool is configured for insertion into a body of a subject and a proximal end is configured to be external to the body of the subject when the distal end is inserted. The apparatus also includes a frame configured to removably and rigidly attach to the proximal end of the tool. The apparatus also includes a plurality of electromagnetic tracking system components, each component comprising an electromagnetic sensor rigidly attached to the frame. The plurality of electromagnetic tracking system components is configured to measure position and orientation of the frame with 6 degrees of freedom.

In a fifth set of embodiments, a method for guided surgical procedures includes producing three-dimensional imagery of a subject relative to spatial fiducials; and, registering an electromagnetic (EM) tracking system relative to the spatial fiducials. The method further includes removably attaching a frame to a proximal portion of a rigid invasive medical tool. Multiple electromagnetic tracking system sensors are rigidly attached to the frame and configured to measure position and orientation of the frame with 6 degrees of freedom. The method still further includes computing, automatically on a processor, a location of an operative portion of the rigid tool based on the position and orientation of the frame and on the EM tracking system and the spatial fiducials. Even further, the method includes using the rigid invasive medical tool in the subject while tracking the position and orientation of the operative portion of the rigid tool relative to the three-dimensional imagery of the subject.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a block diagram that illustrates an example system for image-guided surgical procedures, according to an embodiment;

FIG. 1B and FIG. 1C are block diagrams that illustrate example cross-sections of tools used in the system of FIG. 1A, according to various embodiments;

FIG. 1D is a block diagram that illustrates an example malleable tool for the system of FIG. 1A, according to an embodiment;

FIG. 2 is a flow chart that illustrates an example method for operating the system of FIG.1A, according to an embodiment;

FIG. 3A and FIG. 3B are block diagrams that illustrate longitudinal cross sections of distal ends of a recess in a tool for the system of FIG. 1A, according to various embodiments;

FIG. 3C is a block diagram that illustrates an example transverse cross section of a distal end of a recess for a tool for the system for image-guided surgical procedures, according to an embodiment;

FIG. 4A through FIG. 4G are block diagrams that illustrate example surgical tools configured for the system of FIG. 1A, according to various embodiments;

FIG. 5A through FIG. 5D are block diagrams that illustrate an example brace for the system of FIG. 1A, according to various embodiments;

FIG. 6A is a block diagram that illustrates an example system for image-guided surgical procedures, according to another embodiment;

FIG. 6B is a block diagram that illustrates an example removable frame with an EM sensor suite, according to another embodiment;

FIG. 7 is a flow chart that illustrates an example method for operating the system of FIG.6, according to an embodiment;

FIG. 8 is a block diagram that illustrates a computer system 800 upon which an embodiment of the invention may be implemented; and

FIG. 9 illustrates a chip set 900 upon which an embodiment of the invention may be implemented.

### DETAILED DESCRIPTION

A method, system and apparatus are described for image guided surgical procedures. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5 X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of certain invasive medical tools, such as needles, cannula, spatulas, whirlybirds, curettes, knives, clamps and forceps with attached tubes providing a subcutaneous recess for EM wired sensors. However, the invention is not limited to this context. In other embodiments, other invasive medical tools, including surgical tools, are used, such as flexible or rigid endoscopes, colposcopes, aspirators, biopsy needles, infusion needles, suction tips, catheters, stylets, introducers, robotically controlled arms and implements, and implants such as stents, electrodes, chemical, drug, or energy eluting compounds, among others, with built-in recesses, such as irrigation tubes, or tubes attached internally or externally to provide recesses for EM wired sensors. In some embodiments, the EM sensors are more complex combinations of wired or wireless sensors that provide up to 6 degrees of freedom (DoF) and are attached via a removable frame at the base (proximal portion) of the invasive medical tool, wherein the EM sensor remain outside the body of the subject where space is not so limited and size of the EM sensor combination is not so much an issue.

### 1. OVERVIEW

While EM tracking for image-guided therapy (IGT) has been suggested, the number of invasive medical tools outfitted with EM sensors is limited and the price per tool is high. It is noted here that novel modifications to many invasive medical tools render them suitable for removably inserting an EM sensor, so that the relatively highly priced EM sensors can be reused for multiple tools employed during a surgical procedure. A simple position sensor (3 degrees of freedom, DoF) is small enough to be placed on a subcutaneous portion of a tool, at or near the operative portion of the tool, so that a surgeon can determine where the business end of the tool is, relative to co-registered three dimensional medical imagery. If the sensor is connected by a simple conducting wire, the size and expense and power demands of a wireless connection is avoided in the subcutaneous portion of the tool. Thus, a tool is readily adapted for EM tracking without introducing bulky components that interfere with the surgeon's well practiced movements, and without permanently binding the EM tracking device that renders the tool expensive.

Therefore, in various embodiments, a recess is utilized or added to the tool. The recess is configured to removably engage an electromagnetic tracking system component comprising an electromagnetic sensor and an insulated electrical wire connected to the electromagnetic sensor. Because the sensor is removably engaged, it is possible for the sensor to be in any location along the recess. To ensure that the sensor is at the desired location, e.g., at a known distance from the operative portion of the tool, a viewport is included in some embodiments, to make evident what is in the recess at least at or near the desired location. Thus, in these embodiments, the viewport is configured to reveal a portion of the recess where the electromagnetic sensor is to be disposed.

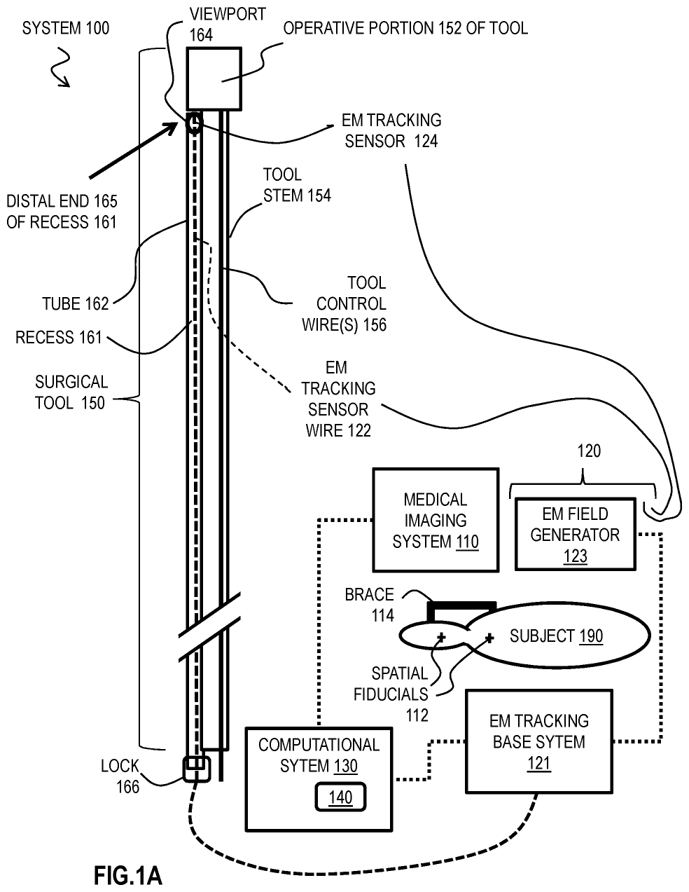

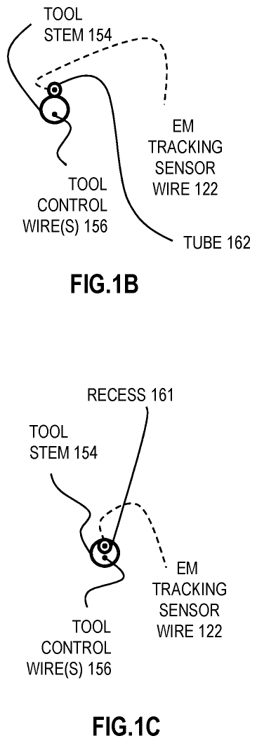

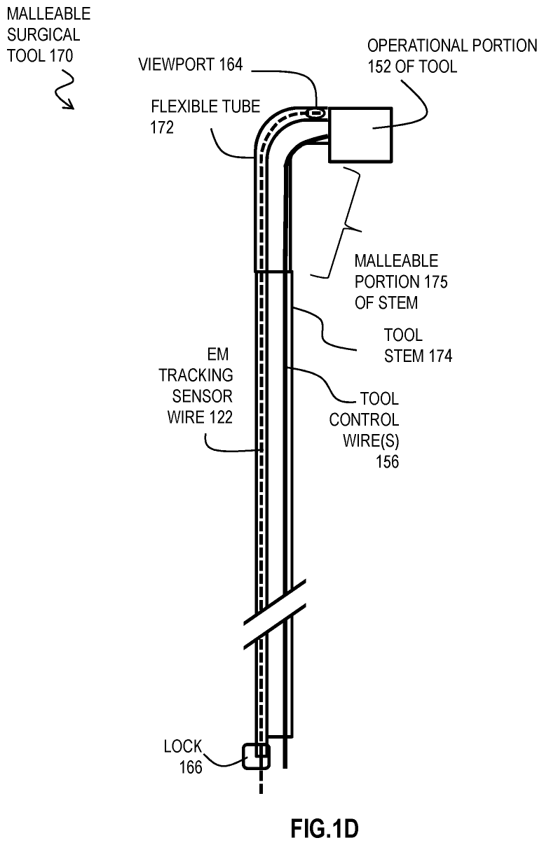

FIG. 1A is a block diagram that illustrates an example system 100 for image-guided surgical procedures, according to an embodiment. The system 100 includes a medical imaging system 110 (such as a CT imaging system or an MRI system). The system 100 also includes an EM tracking system 120 that includes an EM tracking base system 121, an EM field generator 123, an EM tracking sensor 124 connected by an EM tracking sensor wire 122 to the EM tracking base system 121. The sensor 124 is a small sensor for which a 3 dimensional position (3 DoF), but not orientation, is determined by the magnetic flux from the EM field generator 123 measured by the sensor 124. A value for the magnetic flux is indicated by an analog or digital signal transmitted through the wire 122 to the base system 121. The three-dimensional position of the sensor 124 is computed by the base system 121 based on the known position, orientation, and field generated by field generator 123 and on the value of the magnetic flux transmitted by the sensor 124 through the sensor wire 122.

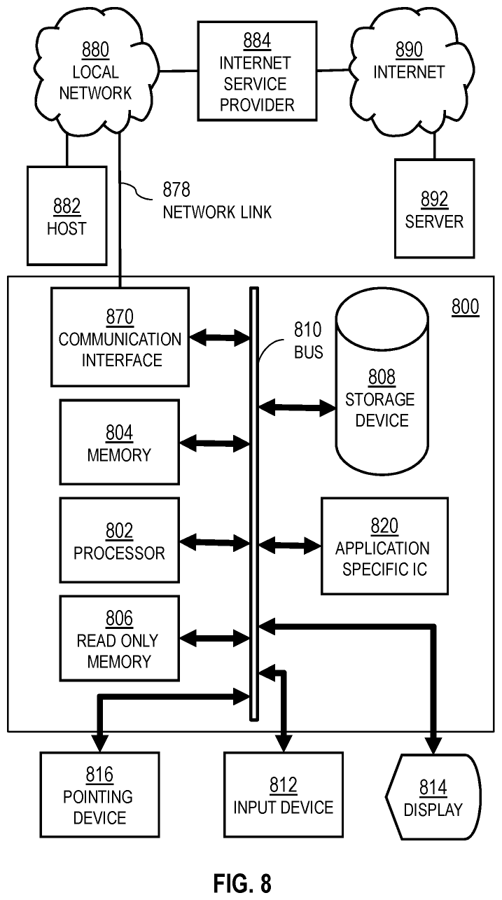

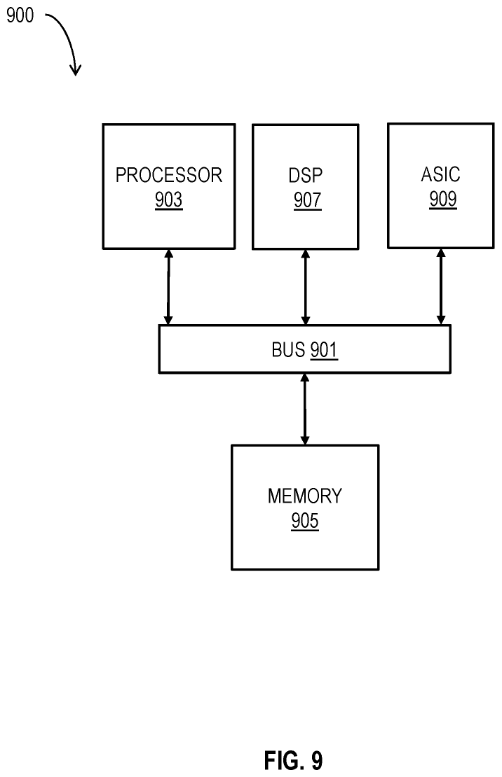

The system 100 also includes a computational system 130, such as computer system 800 described below with reference to FIG. 8 or chip set 900 described below with reference to FIG. 9. The computational system includes a tracking module 140 that determines the position of the sensor 124 relative to three dimensional medical imagery produced by medical imaging system 110. The imagery produced by the system 110 is registered relative to one or more registration markers (also called spatial fiducials 112) disposed in space at known positions, which appear in the imagery. In some embodiments, the EM tracking system 120 is co-registered with the medical imagery produced by the system 110 by sequentially placing an EM tracking sensor, such as sensor 124, at each of the spatial fiducials 112 and recording the positions of the spatial fiducials 112.

During medical procedures, the system operates on a subject 190, such as a currently or formerly living animal, including a human. Although the subject 190 is depicted for purposes of illustrating the use of the system 100, the subject 190 is not part of the system 100. Spatial fiducials 112 are disposed on or near the subject 190. Thus, the system includes registration markers configured to be placed on or near the subject such that the position of the electromagnetic sensor can be determined relative to a three-dimensional scanned medical image of the subject.

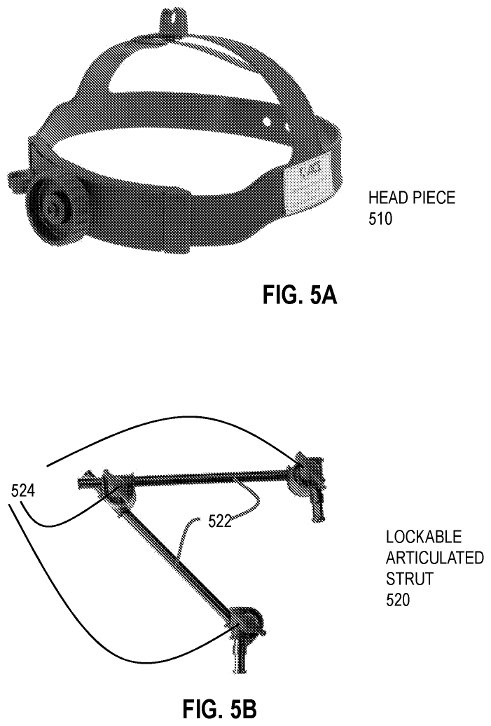

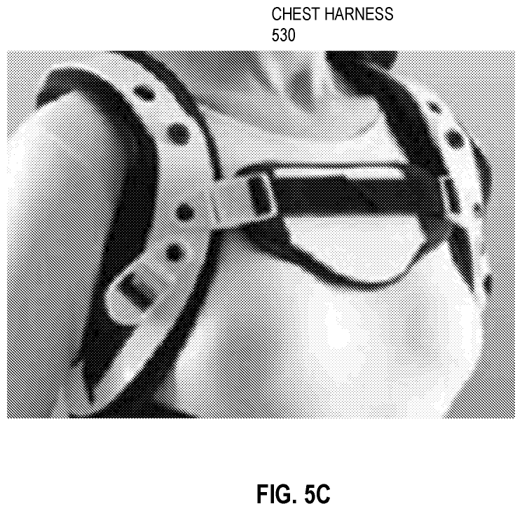

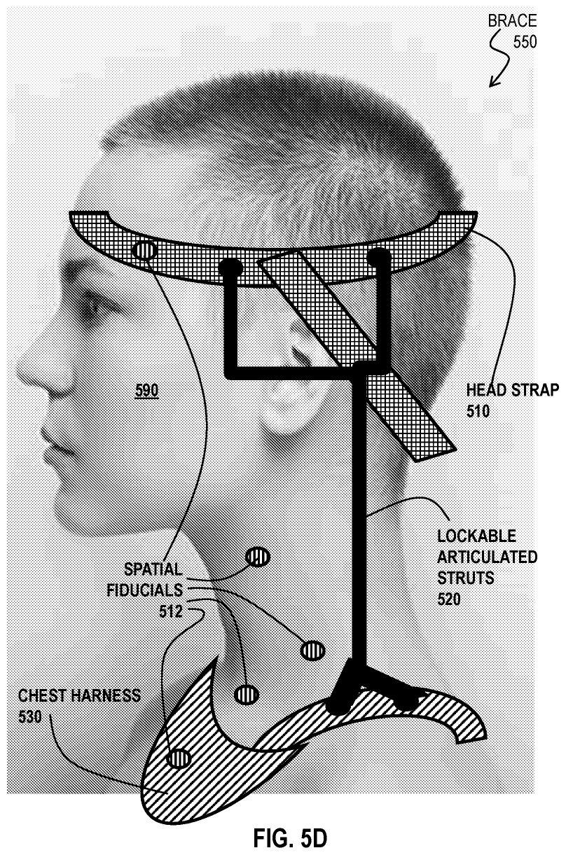

For some medical procedures, such as procedures in the neck or shoulder or hip or wrist or ankle of a human patient, a target volume of the subject includes tissues on either or both sides of one or more joints, including vertebrae. In such circumstances, a brace 114 is included in the system 100 and configured to hold relatively motionless two portions of the subject on opposite sides of a joint of the subject; and, spatial fiducials 112 are disposed on both sides of the joint. The brace 114 is configured to be rigid enough for this purpose and can be adjustable to be reusable with several subjects, as described in more detail below with reference to FIG. 5A through FIG. 5D. The brace 114 is made of materials that are relatively transparent to the medical imaging system 110 and non-interfering with the EM tracking system 120. For example, when the medical imaging system is a CT scanner, the brace 114 is made of materials selected from a group that includes dacron, nylon, polyethylene, polysulfone, polyetherimide, polyetheretherketone, polyphenylsulfone, polychlorotrifluoroethylene, ethylene tetrafluoroethylene, ethylene chlorotrifluoroethylene. For example, when the medical imaging system is an MRI scanner, the brace 114 is made of materials selected from a group that include titanium and gold as well as the above listed materials that are useful with CT scanners.

According to several embodiments, each of one or more commonly used invasive medical tools is configured to support removably positioning a wired EM sensor at or near an operative portion of the tool. Example invasive medical tools include a cannula, a needle, a catheter, a guided curette, a guided ring curette, a spatula, a whirly bird, a gimmick type curette, a 90 degree canal knife, a round knife, a guided giraffe forceps, a clamp, a laparoscope, and an endoscope, among others. A invasive medical tool includes an operative portion 152 configured to interact with a subcutaneous tissue or organ inside a body of the subject, and a stem 154 configured to extend from a stem distal end at a proximal side of the operative portion 152 of the tool to a stem proximal end configured to be manipulated outside the body of the subject. In some embodiments, one or more mechanical or electrical control wires 156 are disposed inside the stem, e.g., to cause the stem 154 to bend or to cause the operative portion 152 to activate, e.g., to grab or clamp some tissue or organ inside the subject or to rotate a lens or lamp of an endoscope or other optical component. Although depicted as straight in FIG. 1A, in other embodiments, the stem 154 has other shapes, such as bent, curved, twisted, tapered or some other shape; and, the tube 162 is correspondingly shaped.

The system 100 includes an invasive medical tool 150 with a recess 161 that extends along the stem 154. The recess 161 is configured to removably engage an electromagnetic tracking system component comprising the electromagnetic sensor 124 and an insulated electrical wire 122 connected to the electromagnetic sensor 124. In the illustrated embodiment, the recess is a lumen of a tube 162 that is attached along the stem. In other embodiments, the recess 161 is provided by an extant lumen in the stem, such as an irrigation tube. The recess extends from a proximal portion of the stem to at least a desired location for the EM sensor. To ensure a surgeon that the operative portion of the tool is as close as possible to a position identified relative to the 3D medical imagery, it is advantageous if the sensor is disposed as close as possible to the operative portion of the tool without interfering with the operation of the tool. Thus, in some embodiments, a distal end 165 of the recess is at the proximal side of the operative portion 152. In some embodiments, this is coincident with a distal end of the stem 154.

The invasive medical tool 150 includes a viewport 164 into the recess 161. The viewport 164 is configured to reveal a portion of the recess 161 where the electromagnetic sensor 124 is to be disposed. In some embodiments, the viewport 164 is a transparent part of the tube 162 or wall of another recess. In some embodiments, the viewport 164 is an opening through a wall of the tube 162 or other recess. In some embodiments, the recess has a diameter between about 0.1 and 0.6 millimeters that is sufficient to accommodate a modern miniature EM tracking sensor and its electrical connecting wire.

In the illustrated embodiment, in order to keep the sensor from shifting its position from the desired location in the viewport during use of the tool 150, the tool 150 includes a lock 166 to hold the wire 122, in place; and, thus keep the sensor 124 in the viewport 164. In some embodiments, the lock 166 is a Luer lock. In some embodiments, the lock also provides a seal at a proximal end of the tool, wherein the proximal end of the tool is configured to remain outside a body of subject into which the tool is inserted, and the seal is configured to pass the insulated electrical wire and to prevent at least one of any of fluid flow around the insulated electrical wire or pressure loss around the insulated electrical wire or movement of the insulated electrical wire.

FIG. 1B and FIG. 1C are block diagrams that illustrate example cross-sections of tools used in the system of FIG. 1A, according to various embodiments. In FIG. 1B, a cylindrical tube 162 with a circular cross section is attached externally to the tool stem 154 to provide a recess 161 for the EM tracking sensor wire 122. Although a tool control wire 156 is depicted inside a lumen of stem 154, in other embodiments the tool control wire 156 is absent or the stem is solid without an interior lumen. Although the tube 162 has a circular cross section, in other embodiments, the tube has a different shaped cross section, such as a polygonal cross section. In some embodiments the tube 162 cross section provides a smooth transition with the cross section of the tool stem 154 without any convex corners, e.g., each of the stem 154 and tube 162 cross sections is rectangular, or the tube 162 cross section is tangent to the stem 154 cross section where the two components are joined. An advantage of avoiding convex corners is to inhibit tissue snagging or capture or infectious agent trapping in the convex corners.

In FIG. 1C, a cylindrical tube with a circular cross section is attached internally to the tool stem 154 to provide the recess 161 for the EM tracking sensor wire 122. Although a tool control wire 156 is depicted inside a lumen of stem 154, in other embodiments the tool control wire 156 is absent. In some of these embodiments, the interior of stem 154, outside recess 161, is solid; and, the recess 161 is formed by grinding out material that would otherwise be inside the stem 154. Although the recess 161 and stem 154 each has a circular cross section, in other embodiments, the recess 161 or stem 154 has a different shaped cross section, such as a polygonal cross section. In some embodiments, the recess 161 has multiple uses; and, is used not just to accommodate the sensor 124 and wire 122 but is also used coincidentally or previously or subsequently, or some combination, for some other purpose such as drainage or aspiration of a subcutaneous volume inside the subject 190.

FIG. 1D is a block diagram that illustrates an example malleable tool for the system of FIG. 1A, according to an embodiment. In this embodiment, the stem 154 is malleable and can be shaped, either by manipulation before subcutaneous insertion, or after insertion, e.g., through the action of one or more tool control wires 156. In this embodiment, a flexible tube 172 is used to provide a recess 161, and the viewport 164 is disposed in the flexible tube 172. Suitable materials for flexible tube 172 include malleable surgical grade ferrous or nonferrous metals/metal alloys or one of the polymer materials listed above, including Dacron, nylon, polyethylene, polysulfone, polyetherimide, polyetheretherketone, polyphenylsulfone, polychlorotrifluoroethylene, ethylene tetrafluoroethylene, ethylene chlorotrifluoroethylene, some of which are transparent. A transparent material offers the advantage of allowing an operator to see that the EM sensor and wire is properly disposed inside the tube. In other embodiments in which the stem is not malleable, the tube 162 can be rigid, whether straight as in FIG. 1A or otherwise shaped, e.g., curved as depicted in FIG. 1D; and, suitable materials for a rigid tube 162 include aluminum, stainless steel, among other surgical grade ferrous or nonferrous metals/metal alloys, and such polymer materials as Dacron, nylon, polyethylene, polysulfone, polyetherimide, polyetheretherketone, polyphenylsulfone, polychlorotrifluoroethylene.

Although processes, structures, equipment, and devices are depicted in FIG. 1A through FIG. 1D, and subsequent structural diagrams, as integral blocks in a particular arrangement for purposes of illustration, in other embodiments one or more processes or structures or equipment or devices, or portions thereof, are arranged in a different manner, or are omitted, or one or more different processes, structures, equipment or devices are included. For example, in some embodiments, EM tracking base system 121 and computational system 130 are combined.

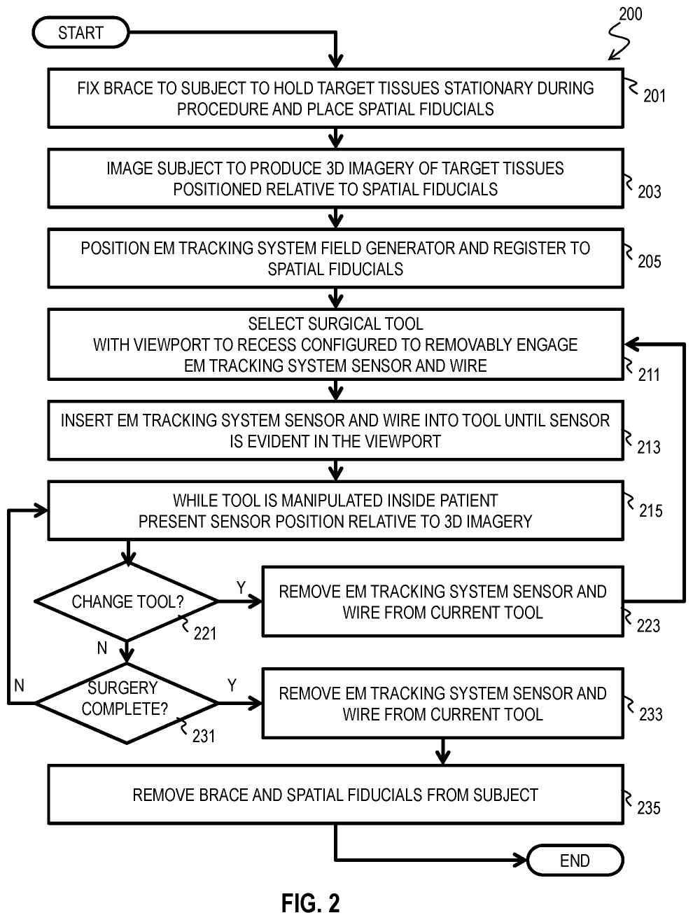

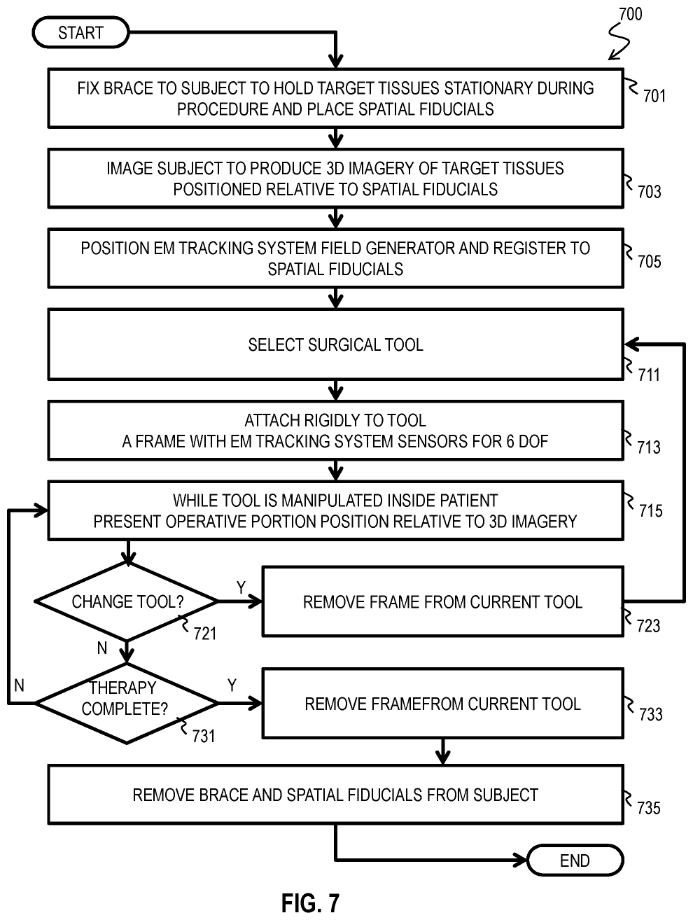

FIG. 2 is a flow chart that illustrates an example method 200 for operating the system of FIG.1A, according to an embodiment. Although steps are depicted in FIG. 2, and in subsequent flowchart FIG. 7, as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 201, a brace 114 is fixed to a subject to hold relatively stationary (motionless) target tissues on either side of a joint in a region of interest (also called target tissues, herein). In addition, multiple spatial fiducials 112 are placed on or near or inside the subject. The spatial fiducials are configured to be detected by the medical imaging system 110 and are accessible to the EM tracking system 120 so that the EM tracking system 120 can be co-registered spatially with the medical imaging system 110. When a joint in the subject is included in the region of interest, the spatial fiducials 112 are distributed to place at least one spatial fiducial 112 on each different side of the joint. In some embodiments, a joint is not included in the region of interest; and, step 201 omits fixing a brace 114 to the subject. Step 201 still includes placing multiple spatial fiducials 112 on or near or inside the subject; but, omits placing spatial fiducials 112 on different sides of the joint.

In step 203, the subject is placed in operational range of the medical imaging system 110; and, the system 110 is operated to produce a three dimensional (3D) medical image of the target tissues and the spatial fiducials 112. The 3D medical imagery is thus registered to the positions of the spatial fiducials 112.

In step 205, The EM tracking system field generator 123 is positioned to produce an EM tracking field in a tracking volume occupied by the target tissues and spatial fiducials 112. In some embodiments, one or more of the spatial fiducials 112 each includes a wired or wireless EM tracking sensor so that the positions of such spatial fiducials 112 are determined by the system 120. If any spatial fiducials 112 do not include a wired or wireless EM tracking sensor, then a wired or wireless EM tracking sensor, such as wired sensor 124, or other tracer provided with the system 120, is placed successively at each such accessible spatial fiducial 112 so that the EM tracking system 120 can determine the position of each such spatial fiducial 112.

In step 211, an invasive medical tool with a recess for engaging an EM tracking wired sensor and wire, and with a viewport into that recess, (e.g., invasive medical tool 150) is selected for use during a medical procedure on the subject 190. In step 213, the EM tracking wired sensor 124 is inserted into the recess until the sensor 124 is evident in the viewport (e.g., visible through the viewport opening or transparent window of tube 162). In some embodiments, step 213 includes locking the sensor 124 or sensor wire 122 in place inside the tool, e.g., inside tool 150 with lock 166. Any locking mechanism known in the art can be used in various embodiments as lock 166, such as using a Luer lock, twist lock, or pressure locking device.

In step 215, while the tool is manipulated inside the subject by a surgeon, a position of the sensor relative to the 3D medical imagery is presented on a display device in view of the surgeon. The surgeon can infer the position of the operative portion of the tool relative to the target tissue based on the position of the EM tracking wired sensor 124 relative to the 3D medical imagery; thus, providing a substantial assist in the way of image guided therapy.

In step 221, it is determined by the surgeon or therapist whether to change the tool. If so, then in step 223 the EM tracking wired sensor 124 is removed from the current tool, e.g., tool 150. In some embodiments, step 223 includes sterilizing the tool or the EM sensor 124 and wire or some combination. The process passes back to step 211 and following steps to select and use a different tool with recess and viewport, if such a different tool is available, e.g. a tool 150 with a different operative portion 152. This loop continues until it is determined in step 231 that the invasive medical procedure on the target tissue is complete. If not complete, the process continues with step 215 to continue to manipulate the tool and display the sensor position relative to the 3D imagery.

If the invasive medical procedure is complete, then the process continues with step 233, in which the EM tracking wired sensor 124 is removed from the current tool, e.g., a tool 150. In some embodiments, step 235 includes sterilizing the tool or the EM sensor 124 and wire or some combination. In step 235 the brace 114, if any, and spatial fiducials 112 are removed from the subject. The process then ends.

Thus, the system 100 and method 200 allow for the reuse of expensive EM tracking components for very precise image guided therapy using multiple inexpensive invasive medical tools that need provide only: a recess for accepting an EM tracking wired sensor with connected wire; and a viewport into the recess.

### 2. EXAMPLE EMBODIMENTS

In this section, several different example embodiments are described for tool 150.

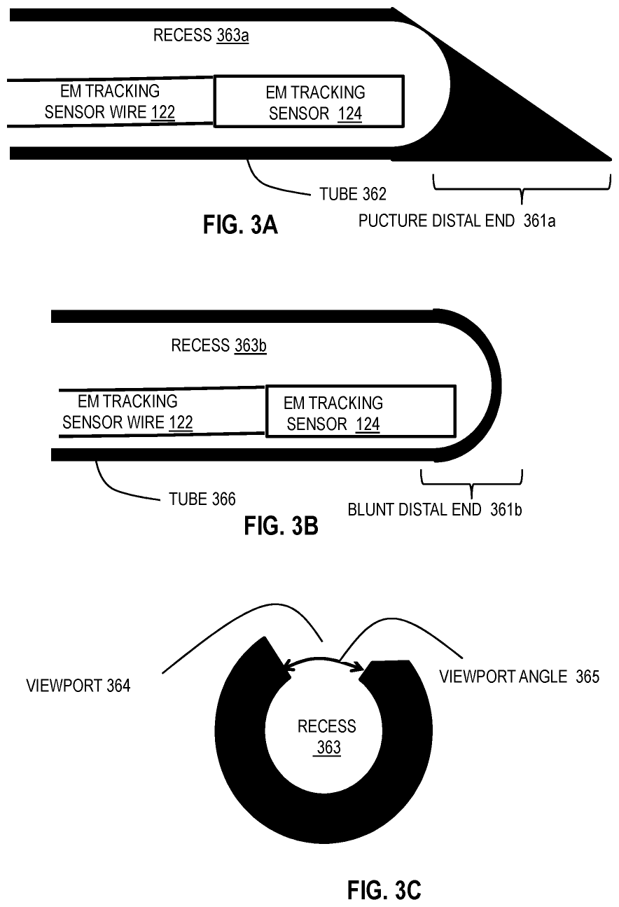

FIG. 3A and FIG. 3B are block diagrams that illustrate longitudinal cross sections for distal ends of a recess in a tool for the system of FIG. 1A, according to various embodiments. In FIG. 3A, a recess 363a is provided by a tube 362 that is attached internally or externally to a stem of a tool. Also depicted in FIG. 3A is an EM tracking wired sensor 124 and the connected wire 122 residing inside the recess 363a. The distal end 361a of the tube 362 is sharp in order to be compatible with a puncturing tool, such as a needle or trocar, and is called a puncture distal end 361a. In FIG. 3B, a recess 367 is provided by a tube 366 that is attached internally or externally to a stem of a tool. Also depicted in FIG. 3B is an EM tracking wired sensor 124 and the connected wire 122 residing inside the recess 363b. The distal end 361b of the tube 366 is blunt in order to be compatible with a non-puncturing tool, such as a curette or spatula, and is called a blunt distal end 361b.

FIG. 3C is a block diagram that illustrates an example transverse cross section of a distal end of a recess 363a or 363b, collectively reference hereinafter as recess 363, for a tool for the system for image-guided surgical procedures, according to an embodiment. In this embodiment, a wall of the recess, whether ground out in an otherwise solid stem, or attached as an external tube 362 or 366, is open or includes a transparent material at least along a portion of the recess length to provide a viewport 364. To prevent an enclosed sensor and wire from escaping through the viewport 364 that uses an opening, the opening is limited to an appropriate range of angles around the cross section, each such angle called for convenience herein a viewport angle. For example, in some embodiments with a recess 163 diameter of about 0.5 mm, the viewport angle is confined to be less than 180 degrees. In some embodiments, the viewport angle is in a range from about 45 degrees to about 135 degrees. In some embodiments, the viewport angle is in a range from 60 degrees to 120 degrees. For example, for a tube with inner diameter of 0.5 mm, a 60 degree opening corresponds to a 0.25 mm opening, through which a sensor would be readily detectably by an operator of the tool. Thus, an opening of about 60 degrees is useful. The largest viewport angle is small enough, such as 135 degrees or 120 degrees, to prevent the EM sensor or wire from passing through the resulting opening.

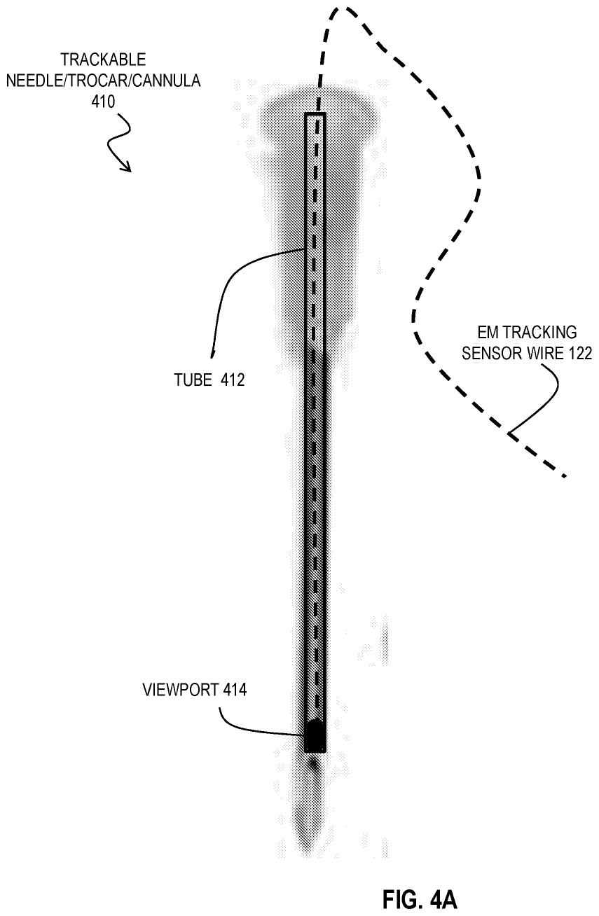

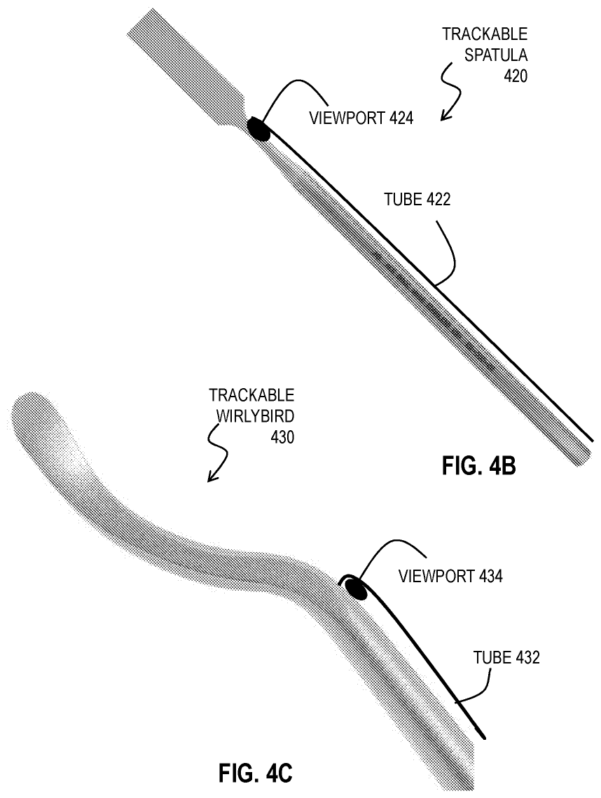

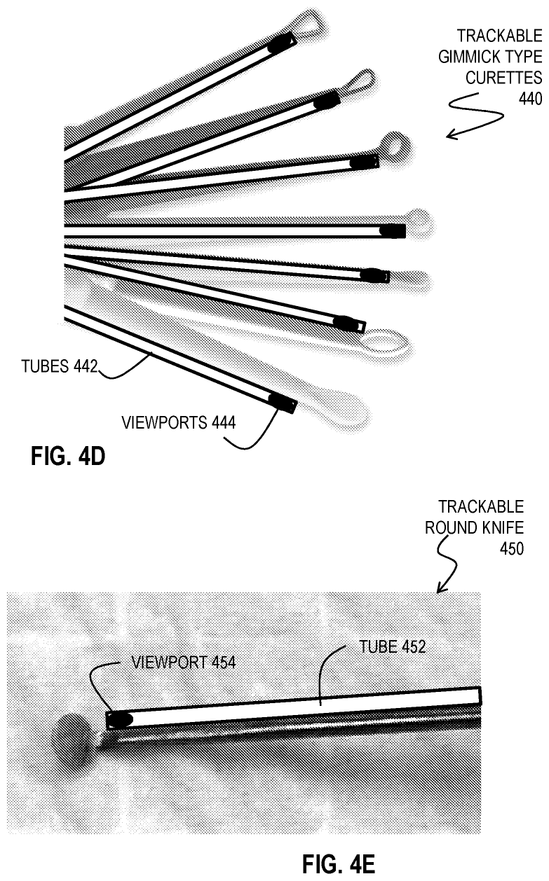

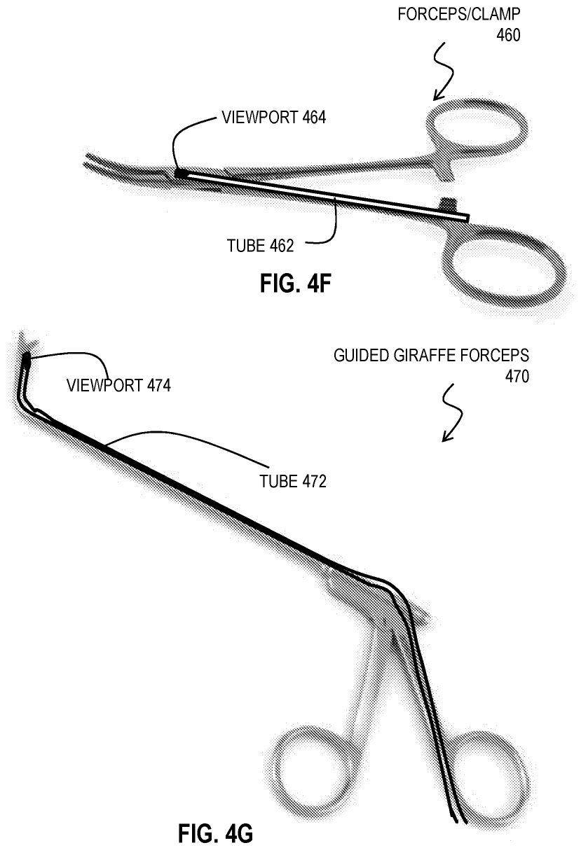

FIG. 4A through FIG. 4G are block diagrams that illustrate example surgical tools configured for the system of FIG. 1A, according to various embodiments. These tools are collectively called trackable tools. In each of the illustrated embodiments, the recess 161 is provided by an embodiment of the attached tube 162. However, in other embodiments, one or more of the recesses provided by attached tubes is replaced by a recess extant or bored into a wall of the tool without attaching a separate tube. In each embodiment, a viewport is included, either in the attached tuber or the stem of the tool or both.

FIG. 4A is a block diagram that illustrates a puncturing tool 410, such as a needle or trocar or cannula, configured with an attached tube 412 with viewport 414, according to an embodiment. In this example embodiment, the tube 412 has a puncturing distal end, as depicted in FIG. 3A. The viewport 414 is near the operative portion made up of the puncturing tip of the tool 410. Also depicted is an EM tracking sensor wire when deployed inside the tube with the sensor evident in the viewport 414. The connection of the wire 122 to the EM tracking base system 120 and the lock 166, if any, are not shown.

FIG. 4B is a block diagram that illustrates a trackable surgical spatula 420 configured with an attached tube 422 with viewport 424, according to an embodiment. In this example embodiment, the tube 422 has a blunt distal end, as depicted in FIG. 3B. The viewport 414 is at the proximal side of the operative portion made up of the flat surface of the tool 410. The sensor 124 and sensor wire 122 connected to the EM tracking base system 120 and the lock 166, if any, are not shown.

FIG. 4C is a block diagram that illustrates a trackable surgical whirlybird 430 configured with an attached tube 432 with viewport 434, according to an embodiment. A conventional whirlybird is a well-known tool used to expand a space inside tissue or between organs or to dissect around corners and rigid ledges, typically using rotational movements. In this example embodiment, the tube 432 has a blunt distal end, as depicted in FIG. 3B. The viewport 434 is at the proximal side of the operative portion made up of the flat curved surface of the tool 430. The sensor 124 and sensor wire 122 connected to the EM tracking base system 120 and the lock 166, if any, are not shown.

FIG. 4D is a block diagram that illustrates multiple trackable gimmick type surgical curettes 440, each configured with an attached tube 442 with viewport 444, according to various embodiments. A conventional curette is a well-known tool used to manipulate tissue or organs. Conventional gimmick type curettes have a variety of different shapes and materials suitable for particular purposes. In this example embodiment, the tube 442 has a blunt distal end, as depicted in FIG. 3B. The viewport 444 is at the proximal side of the operative portion made up of the loop or spoon of the tool 440. The sensor 124 and sensor wire 122 connected to the EM tracking base system 120 and the lock 166, if any, are not shown.

FIG. 4E is a block diagram that illustrates an example trackable surgical round knife 450, configured with an attached tube 452 with viewport 454, according to an embodiment. A conventional round knife is a well-known tool used to cut tissue. In this example embodiment, the tube 452 has a blunt distal end, as depicted in FIG. 3B. The viewport 454 is at the proximal side of the operative portion made up of the disk-shaped knife with a cutting edge along most of a circumference of the disk. The sensor 124 and sensor wire 122 connected to the EM tracking base system 120 and the lock 166, if any, are not shown.

FIG. 4F is a block diagram that illustrates an example trackable surgical forceps/clamp 460, configured with an attached tube 462 with viewport 464, according to an embodiment. A conventional forceps/clamp is a well-known tool used to grasp and hold tissue without continued exertion by a surgeon; or is used for removal/extraction of tissue and other materials; or is used for placement of grafting materials or implants. In this example embodiment, the tube 462 has a blunt distal end, as depicted in FIG. 3B. The viewport 464 is at the proximal side of the operative portion made up of the prongs of the forceps to the distal side of a fulcrum or pivot for the prongs. The sensor 124 and sensor wire 122 connected to the EM tracking base system 120 and the lock 166, if any, are not shown.

FIG. 4G is a block diagram that illustrates an example trackable giraffe forceps 470, configured with an attached tube 472 with viewport 474, according to an embodiment. A conventional giraffe forceps is a well-known tool used to grasp and hold tissue in limited or predefined space, e.g., for working in the frontal and maxillary sinus. In this example embodiment, the tube 472 has a blunt distal end, as depicted in FIG. 3B. The viewport 474 is at the proximal side of the operative portion made up of the prongs of the forceps to the distal side of a fulcrum or pivot for the prongs. The sensor 124 and sensor wire 122 connected to the EM tracking base system 120 and the lock 166, if any, are not shown.

FIG. 5A through FIG. 5D are block diagrams that illustrate an example brace 114 for the system of FIG. 1A, according to various embodiments. This brace is appropriate for therapies applied in the head and neck, such as the brain and spinal cord, or mouth and throat, of a subject. FIG. 5A is a perspective view that illustrates an example head piece 510 used in some embodiments.

FIG. 5B is a perspective view that illustrates an example lockable articulated strut 520 used in some embodiments. The strut 520 includes several rigid segments 522, such as rods, of which two or more are connected at mechanical joints 524 that can be loosened to change an angle between segments or tightened to hold a given angle between segments. In some embodiments, each joint 524 has a universally rotatable ball joint to accommodate for head positioning, and an, at least bilateral, attachment point for the struts 522.

FIG. 5C is a perspective view that illustrates an example chest harness 530 in use, according to some embodiments. The harness 530 serves as a chest/shoulder stabilizer and is similar to the upper portion of a Thoracic Lumbar Sacral Orthosis (TLSO) brace used for spinal stabilization.

FIG. 5D is a block diagram that illustrates an example brace 550 for head and neck image guided therapy, according to an embodiment. Although a subject 590 is depicted for purposes of illustration, the subject 590 is not part of the brace 550. The brace 550 includes the head strap 510 and the chest harness 530, each rigidly connected to the lockable articulated struts 520 when the latter are in locked status. The three-point fixation headpiece goes around a forehead and crown of a head of the subject, with an optional accessory band over the occiput (base of skull). Ratchets to tighten the head piece are not shown. Black ovals on the headpiece 510 represent anchor points for attaching the lockable articulated struts 520 onto the headpiece 510. Black ovals on the chest harness 530 represent anchor points for attaching the lockable articulated struts 520 onto chest harness. The struts 520 are attached bilaterally (two opposite sides of the head) in some embodiments; but, can be used unilaterally in other embodiments. At least the struts 520 are radiolucent or non-metalic, preventing artifacts when CT or MRI scans, respectively, are taken of the subject.

The vertically hatched dots represent spatial fiducials 512 (that serve as imaging markers) on the headpiece 510, the chest harness 530, and on the skin of the subject 590, that would be used to ensure that the ultimate correlation of the scan lined up with the subject positioning.

### 3. ALTERNATIVE TOOL AND METHOD

In the embodiments described in this section, instead of a recess and viewport, a subcutaneous rigid tool is outfitted with a removable frame on the proximal portion of the tool stem to which is attached an EM sensor combination that provides six (6) degrees of freedom (DoF). This configuration, while different and more complex than the system of FIG. 1A, offers similar advantages in allowing reuse of relatively expensive EM tracking components on a variety of otherwise conventional invasive medical tools, such as invasive medical tools. Because the EM sensors are removably attached to the proximal portion of the stem that resides outside the body of the subject, the EM components can be substantially larger. This enables the frame to hold enough sensors for 6 DoF and even a wireless connection to the EM tracking base system.

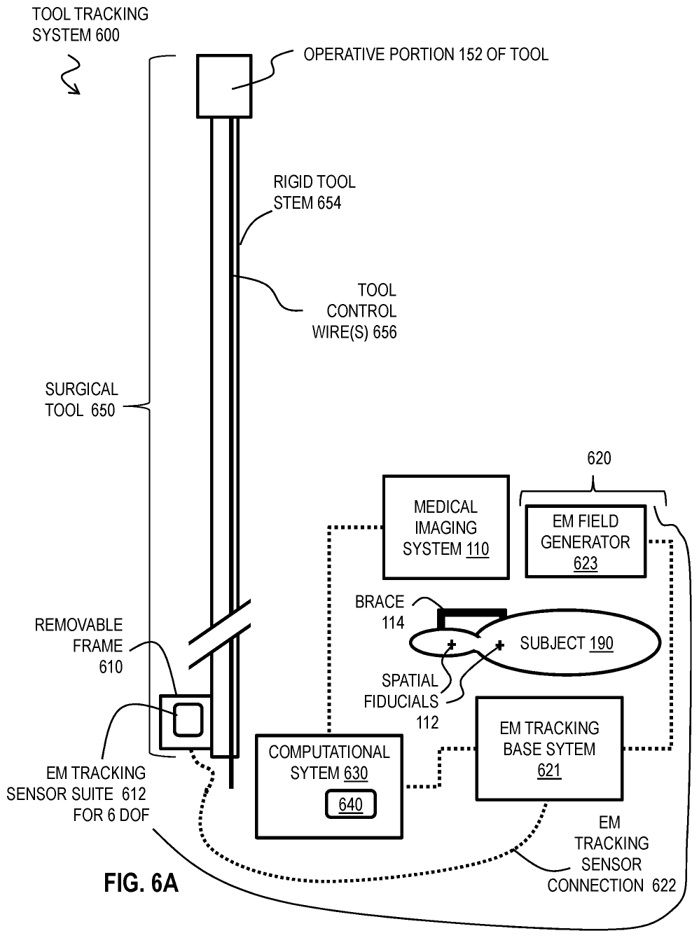

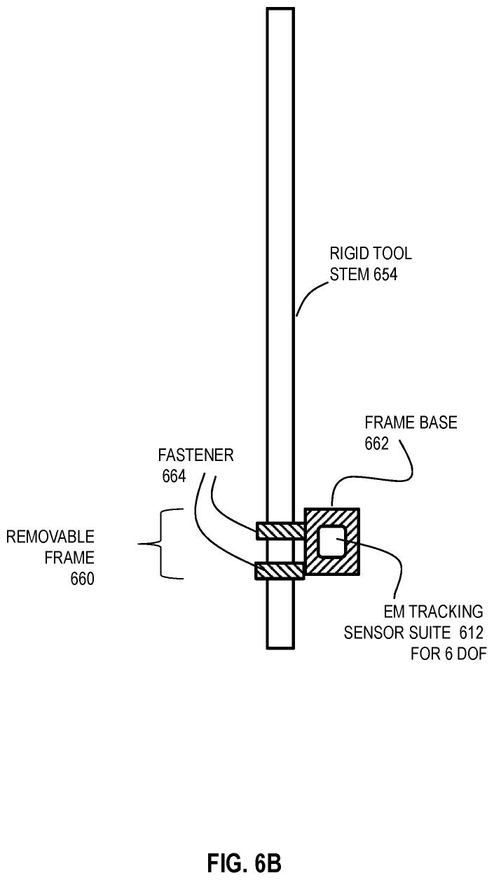

FIG. 6A is a block diagram that illustrates an example system 600 for image-guided surgical procedures, according to another embodiment. The system 600 includes a medical imaging system 110 (such as a CT imaging system or a MRI system). The system 600 also includes an EM tracking system 620 that includes an EM tracking base system 621, an EM field generator 623, an EM tracking sensor suite 612 that provides 6 DoF connected by a wired or wireless EM tracking sensor connection 622 to the EM tracking base system 621. The three dimensional position and orientation of the sensor suite 612 is computed by the base system 621 based on the known position, orientation, and field generated by field generator 623 and on the values of the measurements transmitted by the sensor suite 612 as analog or digital signals through the sensor connection 622. The sensor suite 612 is rigidly attached to a frame 610 that is configured to be rigidly and removably attached to each of one or more conventional invasive medical tools. FIG. 6B is a block diagram that illustrates an example removable frame 660 with an EM sensor suite, according to another embodiment. The frame includes a base 662, to which the EM tracking sensor suite 612 is rigidly fixed, and one or more fasteners 664, such as spring clamps, screw clamps, band clamps 664, or other fastener, configured to rigidly and removably attach the base 662 to a proximal end of a stem 654 of the invasive medical tool 650.

The system 600 also includes a computational system 630, such as computer system 800 described below with reference to FIG. 8 or chip set 900 described below with reference to FIG. 9. The computational system includes a tracking module 640 that determines the position and orientation of the sensor suite 612. This is used with information about the spatial offset of the operative portion of the tool from the frame to provide the position and orientation of the operative portion 152 of the tool relative to three dimensional (3D) medical imagery produced by medical imaging system 110. In some embodiments, the EM tracking system 620 is co-registered with the medical imagery produced by the system 110 by sequentially placing an EM tracking sensor, such as sensor 124 or suite 612, at each of the spatial fiducials 112 and recording the positions of the spatial fiducials 112.

Although the subject 190 is depicted for purposes of illustrating the use of the system 600, the subject 190 is not part of the system 600.

The frame 610 is configured to removably and rigidly attach to a proximal portion of each of one or more invasive medical tools. As described above, an invasive medical tool includes an operative portion 152 configured to arrive at or interact with a subcutaneous tissue or organ inside a body of the subject, and a stem 154 configured to extend from a stem distal end at a proximal side of the operative portion 152 of the tool to a stem proximal portion configured to be manipulated outside the body of the subject. In some embodiments, one or more mechanical or electrical control wires 156 are disposed inside the stem, e.g., to cause the operative portion 152 to activate, e.g., to grab or clamp some tissue or organ inside the subject or to rotate a lens or lamp of an endoscope or other optical component. Although depicted as straight in FIG. 1A, in other embodiments the stem 154 has other shapes, such as bent, curved, twisted, tapered or some other shape.

The frame includes a base to which the sensor suite is attached, such as the Six degree of freedom (6 DoF) Sensor for the NDI Aurora ac tracking system. The frame also includes one or more adjustable fasteners configured to removably attach the frame rigidly to the proximal portion of one or more invasive medical tools, as depicted in FIG. 6B. In various embodiments, a 6 DOF tracking sensor is removably attached to the non-working end of instruments using two point fixation, aligned to depressed or extruded points on the instrument and using a sliding pressure clasp. In such embodiments, the sensor is attached and detached during the procedure, due to the two-point fixation, without loss of fidelity as long as the instrument continues to be registered. The two point fixation can be on the same side, on opposing sides, or offset radially along the body of the instrument, and will be the same on all instrumentation to accommodate for a sensor that has the same female mate end corresponding to the 2 point male fixation end.

FIG. 7 is a flow chart that illustrates an example method for operating the system of FIG.6, according to an embodiment. In step 701, as in step 201, a brace 114 is fixed to a subject to hold relatively stationary (motionless) target tissues on either side of a joint in a region of interest (also called target tissues, herein). In addition, multiple spatial fiducials 112 are placed on or near or inside the subject. The spatial fiducials are configured to be detected by the medical imaging system 110 and are accessible to the EM tracking system 620 so that the EM tracking system 620 can be co-registered spatially with the medical imaging system 110. When a joint in the subject is included in the region of interest, the spatial fiducials 612 are distributed to place at least one spatial fiducial 612 on each different side of the joint. In some embodiments, a joint is not included in the region of interest; and, step 701 omits fixing a brace 114 to the subject. Step 701 still includes placing multiple spatial fiducials 112 on or near or inside the patient; but, is released from placing spatial fiducials 112 on different sides of the joint.

In step 703, the subject is placed in operational range of the medical imaging system 110; and, the system 110 is operated to produce a three dimensional (3D) medical image of the target tissues and the spatial fiducials 612. The 3D medical imagery is thus registered to the positions of the spatial fiducials 612.

In step 705, The EM tracking system field generator 623 is positioned to produce an EM tracking field in a tracking volume occupied by the target tissues and spatial fiducials 112, as in step 205.

In step 711, one or more invasive medical tools (e.g., invasive medical tool 650) are selected for use during a medical procedure on the subject 690. In step 713, the frame 610 with the EM tracking sensor suite 612 is removably attached to a proximal portion of the stem of the selected one or more invasive medical tools. The distance and direction from the base of the frame where the sensor suite is attached to the operative portion of the tool is measured, e.g., with a manual device, such as a caliper or compass, or automatically using a tracer of the EM tracking system 120, and saved for use in a later step, e.g., on a computer-readable medium in computational system 630.

In step 715, while the one or more tools are manipulated inside the subject by a surgeon, a position of the operative portion 152 relative to the 3D medical imagery is presented on a display device in view of the surgeon. The module 140 determines the position of the operative portion of the tool relative to the 3D medical imagery based on the position and orientation of the sensor suite 612 and the known position of the fiducials and the distance and direction from the base of the frame where the sensor suite is attached to the operative portion of the tool. A position of the operative portion 152 of the tool 650 relative to the 3D medical imagery is presented on a display device in view of the surgeon; thus, providing a substantial assist in the way of image guided therapy.

In step 721, it is determined by the surgeon or therapist whether to change one or more tools. If so, then in step 723 the frame is detached from the current tool, e.g., tool 650. In some embodiments, step 723 includes cleaning or sterilizing the frame. The process passes back to step 711 and following steps to select and attach the frame to a different tool, if such a tool is amenable to the attachment mechanism of the frame, e.g. a tool 650 with a different operative portion 652. This loop continues until it is determined in step 731 that the invasive medical on the target tissue is complete. If not complete, the process continues with step 715 to continue to manipulate the tool and display the sensor position relative to the 3D imagery.

If the invasive medical procedure is complete, then the process continues with step 733, in which the frame is detached from the current tool, e.g., a tool 650. In some embodiments, step 733 includes cleaning or sterilizing the frame. In step 735 the brace 114, if any, and spatial fiducials 112 are removed from the subject. The process then ends.

Thus, the system 600 and method 700 allow for the reuse of expensive EM tracking components for very precise image guided therapy using multiple inexpensive invasive medical tools and a reusable frame rigidly attached to an EM tracking sensor suite that provides 6 DoF.

### 4. COMPUTATIONAL HARDWARE OVERVIEW

FIG. 8 is a block diagram that illustrates a computer system 800 upon which an embodiment of the invention may be implemented. Computer system 800 includes a communication mechanism such as a bus 810 for passing information between other internal and external components of the computer system 800. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 800, or a portion thereof, constitutes a means for performing one or more steps of one or more methods described herein.

A sequence of binary digits constitutes digital data that is used to represent a number or code for a character. A bus 810 includes many parallel conductors of information so that information is transferred quickly among devices coupled to the bus 810. One or more processors 802 for processing information are coupled with the bus 810. A processor 802 performs a set of operations on information. The set of operations include bringing information in from the bus 810 and placing information on the bus 810. The set of operations also typically include comparing two or more units of information, shifting positions of units of information, and combining two or more units of information, such as by addition or multiplication. A sequence of operations to be executed by the processor 802 constitutes computer instructions.

Computer system 800 also includes a memory 804 coupled to bus 810. The memory 804, such as a random access memory (RAM) or other dynamic storage device, stores information including computer instructions. Dynamic memory allows information stored therein to be changed by the computer system 800. RAM allows a unit of information stored at a location called a memory address to be stored and retrieved independently of information at neighboring addresses. The memory 804 is also used by the processor 802 to store temporary values during execution of computer instructions. The computer system 800 also includes a read only memory (ROM) 806 or other static storage device coupled to the bus 810 for storing static information, including instructions, that is not changed by the computer system 800. Also coupled to bus 810 is a non-volatile (persistent) storage device 808, such as a magnetic disk or optical disk, for storing information, including instructions, that persists even when the computer system 800 is turned off or otherwise loses power.

Information, including instructions, is provided to the bus 810 for use by the processor from an external input device 812, such as a keyboard containing alphanumeric keys operated by a human user, or a sensor. A sensor detects conditions in its vicinity and transforms those detections into signals compatible with the signals used to represent information in computer system 800. Other external devices coupled to bus 810, used primarily for interacting with humans, include a display device 814, such as a cathode ray tube (CRT) or a liquid crystal display (LCD), for presenting images, and a pointing device 816, such as a mouse or a trackball or cursor direction keys, for controlling a position of a small cursor image presented on the display 814 and issuing commands associated with graphical elements presented on the display 814.

In the illustrated embodiment, special purpose hardware, such as an application specific integrated circuit (IC) 820, is coupled to bus 810. The special purpose hardware is configured to perform operations not performed by processor 802 quickly enough for special purposes. Examples of application specific ICs include graphics accelerator cards for generating images for display 814, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 800 also includes one or more instances of a communications interface 870 coupled to bus 810. Communication interface 870 provides a two-way communication coupling to a variety of external devices that operate with their own processors, such as printers, scanners and external disks. In general, the coupling is with a network link 878 that is connected to a local network 880 to which a variety of external devices with their own processors are connected. For example, communication interface 870 may be a parallel port or a serial port or a universal serial bus (USB) port on a personal computer. In some embodiments, communications interface 870 is an integrated services digital network (ISDN) card or a digital subscriber line (DSL) card or a telephone modem that provides an information communication connection to a corresponding type of telephone line. In some embodiments, a communication interface 870 is a cable modem that converts signals on bus 810 into signals for a communication connection over a coaxial cable or into optical signals for a communication connection over a fiber optic cable. As another example, communications interface 870 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 870 sends and receives electrical, acoustic or electromagnetic signals, including infrared and optical signals, that carry information streams, such as digital data.

The term computer-readable medium is used herein to refer to any medium that participates in providing information to processor 802, including instructions for execution. Such a medium may take many forms, including, but not limited to, non-volatile media, volatile media and transmission media. Non-volatile media include, for example, optical or magnetic disks, such as storage device 808. Volatile media include, for example, dynamic memory 804. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 802, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 802, except for carrier waves and other signals.

Logic encoded in one or more tangible media includes one or both of processor instructions on a computer-readable storage media and special purpose hardware, such as ASIC 820.

Network link 878 typically provides information communication through one or more networks to other devices that use or process the information. For example, network link 878 may provide a connection through local network 880 to a host computer 882 or to equipment 884 operated by an Internet Service Provider (ISP). ISP equipment 884 in turn provides data communication services through the public, world-wide packet-switching communication network of networks now commonly referred to as the Internet 890. A computer called a server 892 connected to the Internet provides a service in response to information received over the Internet. For example, server 892 provides information representing video data for presentation at display 814.

The invention is related to the use of computer system 800 for implementing the techniques described herein. According to one embodiment of the invention, those techniques are performed by computer system 800 in response to processor 802 executing one or more sequences of one or more instructions contained in memory 804. Such instructions, also called software and program code, may be read into memory 804 from another computer-readable medium such as storage device 808. Execution of the sequences of instructions contained in memory 804 causes processor 802 to perform the method steps described herein. In alternative embodiments, hardware, such as application specific integrated circuit 820, may be used in place of or in combination with software to implement the invention. Thus, embodiments of the invention are not limited to any specific combination of hardware and software.

The signals transmitted over network link 878 and other networks through communications interface 870, carry information to and from computer system 800. Computer system 800 can send and receive information, including program code, through the networks 880, 890 among others, through network link 878 and communications interface 870. In an example using the Internet 890, a server 892 transmits program code for a particular application, requested by a message sent from computer 800, through Internet 890, ISP equipment 884, local network 880 and communications interface 870. The received code may be executed by processor 802 as it is received, or may be stored in storage device 808 or other non-volatile storage for later execution, or both. In this manner, computer system 800 may obtain application program code in the form of a signal on a carrier wave.

Various forms of computer readable media may be involved in carrying one or more sequence of instructions or data or both to processor 802 for execution. For example, instructions and data may initially be carried on a magnetic disk of a remote computer such as host 882. The remote computer loads the instructions and data into its dynamic memory and sends the instructions and data over a telephone line using a modem. A modem local to the computer system 800 receives the instructions and data on a telephone line and uses an infra-red transmitter to convert the instructions and data to a signal on an infra-red a carrier wave serving as the network link 878. An infrared detector serving as communications interface 870 receives the instructions and data carried in the infrared signal and places information representing the instructions and data onto bus 810. Bus 810 carries the information to memory 804 from which processor 802 retrieves and executes the instructions using some of the data sent with the instructions. The instructions and data received in memory 804 may optionally be stored on storage device 808, either before or after execution by the processor 802.

FIG. 9 illustrates a chip set 900 upon which an embodiment of the invention may be implemented. Chip set 900 is programmed to perform one or more steps of a method described herein and includes, for instance, the processor and memory components described with respect to FIG. 8 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 900, or a portion thereof, constitutes a means for performing one or more steps of a method described herein.

In one embodiment, the chip set 900 includes a communication mechanism such as a bus 901 for passing information among the components of the chip set 900. A processor 903 has connectivity to the bus 901 to execute instructions and process information stored in, for example, a memory 905. The processor 903 may include one or more processing cores with each core configured to perform independently. A multi-core processor enables multiprocessing within a single physical package. Examples of a multi-core processor include two, four, eight, or greater numbers of processing cores. Alternatively, or in addition, the processor 903 may include one or more microprocessors configured in tandem via the bus 901 to enable independent execution of instructions, pipelining, and multithreading. The processor 903 may also be accompanied with one or more specialized components to perform certain processing functions and tasks such as one or more digital signal processors (DSP) 907, or one or more application-specific integrated circuits (ASIC) 909. A DSP 907 typically is configured to process real-world signals (e.g., sound) in real time independently of the processor 903. Similarly, an ASIC 909 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 903 and accompanying components have connectivity to the memory 905 via the bus 901. The memory 905 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 905 also stores the data associated with or generated by the execution of one or more steps of the methods described herein.

### 5. ALTERNATIVES, DEVIATONS AND MODIFICATIONS

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

### 6. REFERENCES

The entire contents of each of the following references is hereby incorporated by reference as if fully set forth herein, except for terminology inconsistent with that used herein.
Franz, A. M., T. Haidegger, W. Birkfellner, K. Cleary, T. Peters, and L. Maler-Hein, “Electromagnetic Tracking in Medicine—A review of Technology, Validation and Applications.” IEEE Transactions on Medical Imaging, Vol. 33(8), pp. 1702-1725, August 2014.

What is claimed is:

1. An apparatus comprising:
an operative portion that is configured to perform some invasive medical action;
a stem having a distal end attached to a proximal end of the operative portion and a proximal end configured to be held outside a subject during the invasive medical action;
a recess along the stem, the recess configured to removably engage an electromagnetic tracking system component comprising an electromagnetic sensor and an insulated electrical wire connected to the electromagnetic sensor; and,
a viewport configured to reveal a portion of the recess where the electromagnetic sensor is to be disposed.

2. The apparatus as recited in claim 1, wherein the recess is a cylindrical axial space between a distal end of the stem and a proximal end of the stem.

3. The apparatus as recited in claim 2, wherein the viewport is a break in a wall of the cylindrical space, such that, at the location of the break, a wall of the cylindrical space is more than 180 degrees around an axis of the cylindrical space and less than 330 degrees around the axis of the cylindrical space.

4. The apparatus as recited in claim 2, wherein a cross section of the cylindrical space is a polygon.

5. The apparatus as recited in claim 2, wherein a cross section of the cylindrical space is a circle.

6. The apparatus as recited in claim 1, further comprising a seal at a proximal end of the tool, wherein the proximal end of the tool is configured to remain outside a body of subject into which the tool is inserted, and the seal is configured to pass the insulated electrical wire and to prevent at least one of any of fluid flow around the insulated electrical wire or pressure loss around the insulated electrical wire or movement of the insulated electrical wire.

7. The apparatus as recited in claim 1, wherein a distal end of the tube is beveled to promote piercing of tissue.

8. The apparatus as recited in claim 1, wherein a distal end of the tube is blunt, to inhibit piercing of tissue.

9. The apparatus as recited in claim 1, wherein the recess is a lumen of a flexible tube that is fixed to the stem.

10. The apparatus as recited in claim 9, wherein the flexible tube is inside a lumen of the stem.

11. The apparatus as recited in claim 9, wherein the flexible tube is outside the stem.

12. The apparatus as recited in claim 1, wherein the recess is an irrigation tube that is otherwise configured for aspiration or suction of subcutaneous tissue at the operational portion.

13. The apparatus as recited in claim 1, wherein the viewport is a transparent portion at a distal end of the recess.

14. The apparatus as recited in claim 1, wherein the recess has an inner diameter in a range from 0.1 mm to 0.5 mm.

15. The apparatus as recited in claim 6, wherein the seal is a Luer lock.

16. The apparatus as recited in claim 1, wherein the stem is malleable.

17. The apparatus as recited in claim 1, wherein the viewport is disposed adjacent to a proximal side of the operational portion.

18. The apparatus as recited in claim 9 wherein the viewport is disposed adjacent to a distal end of the stem.

19. A system comprising the apparatus of claim 1 and the electromagnetic tracking system comprising a magnetic field generator configured to be disposed outside a subject into which the tool is to be inserted and the electromagnetic sensor and the insulated electrical wire, wherein the sensor and a portion of the insulated electrical wire are removably disposed inside the recess.

20. The system as recited in claim 19 further comprising a brace configured to hold motionless two portions of the subject on opposite sides of a joint of the subject.

21. The system as recited in claim 20, wherein the joint is the neck and the brace further comprises a band configured to be strapped to a head of a the subject, a chest plate configured to be strapped to a chest of the subject, and an extension arm configured to be rigidly attached to the head band and the chest plate.

22. The system as recited in claim 19, further comprising a plurality of registration markers configured to be placed on or near or inside the subject such that the position of the electromagnetic sensor can be determined relative to a three-dimensional scanned medical image of the subject.

23. A method for guided surgical procedures, comprising:
producing three-dimensional imagery of a subject relative to spatial fiducials;
registering an electromagnetic (EM) tracking system relative to the spatial fiducials;
inserting a wired sensor and wire of the EM tracking system into a first recess with a first viewport of a first invasive medical tool until the wired sensor is disposed at a desired location and evident through the viewport; and
using the first invasive medical tool in the subject while tracking the position of the wired sensor relative to the three-dimensional imagery of the subject based on the EM tracking system and the spatial fiducials.

24. The method for guided surgical procedures as recited in claim 21, further comprising:
selecting a different second invasive medical tool;
removing the wired sensor and wire of the EM tracking system from the first recess of the first invasive medical tool;
inserting the wired sensor and wire of the EM tracking system into a second recess with a second viewport of the second invasive medical tool until the wired sensor is disposed at a desired location and evident through the second viewport; and
using the second invasive medical tool in the subject while tracking the position of the wired sensor relative to the three-dimensional imagery of the subject based on the EM tracking system and the spatial fiducials.

25. The method for guided surgical procedures as recited in claim 23, further comprising, before producing three-dimensional imagery of the subject:
attaching a brace to the subject to immobilize a joint of the subject; and
attaching at least one spatial fiducial on each of at least two different sides of the joint.

26. An apparatus for guided surgical procedures, comprising:
a rigid surgical tool, wherein a distal end of the tool is configured for insertion into a body of a subject and a proximal end is configured to be external to the body of the subject when the distal end is inserted;
a frame configured to removably and rigidly attach to the proximal end of the subcutaneous insertion tool; and
a plurality of electromagnetic tracking system components, each component comprising an electromagnetic sensor rigidly attached to the frame,
wherein the plurality of electromagnetic tracking system components are configured to measure position and orientation of the frame with 6 degrees of freedom.

27. A method for guided surgical procedures, comprising:
producing three-dimensional imagery of a subject relative to spatial fiducials;
registering an electromagnetic (EM) tracking system relative to the spatial fiducials;
removably attaching a frame to a proximal portion of a rigid invasive medical tool, wherein a plurality of electromagnetic tracking system sensors is rigidly attached to the frame and configured to measure position and orientation of the frame with 6 degrees of freedom;
computing, automatically on a processor, a location of an operative portion of the rigid tool based on the position and orientation of the frame and on the EM tracking system and the spatial fiducials; and
using the rigid invasive medical tool in the subject while tracking the position and orientation of the operative portion of the rigid tool relative to the three-dimensional imagery of the subject.
